(19) United States
(12) Patent Application Publication
(10) Pub. No.: US 2022/0387742 A1
(43) Pub. Date: Dec. 8, 2022

(54) PATIENT TRACHEOSTOMY COLLAR

(71) Applicants: Carolyn Kesler-Carter, Sandy, UT (US); Zachery D. Henderson, South Weber, UT (US); Daniel H. Stuart, Layton, UT (US); Theodore R. Espiritu, JR., West Valley City, UT (US)

(72) Inventors: Carolyn Kesler-Carter, Sandy, UT (US); Zachery D. Henderson, South Weber, UT (US); Daniel H. Stuart, Layton, UT (US); Theodore R. Espiritu, JR., West Valley City, UT (US)

(21) Appl. No.: 17/805,395

(22) Filed: Jun. 3, 2022

Related U.S. Application Data
(60) Provisional application No. 63/197,075, filed on Jun. 4, 2021.

(51) Int. Cl. A61M 16/04

(52) U.S. Cl. CPC A61M 16/0497; A61M 16/0465; A61M 2205/59

(57) ABSTRACT

A tracheostomy collar is described herein having a number of features providing ease of use in caring for a tracheostomy and securing a tracheostomy tube inserted therein.

### RELATED APPLICATIONS

This application claims priority to U.S. Provisional Patent Application No. 63/197,075, filed on Jun. 4, 2021, and titled PATIENT TRACHEOSTOMY COLLAR, which is incorporated by reference herein in its entirety.

### BACKGROUND

Tracheostomy collars are used to properly maintain placement of trachea breathing tubes. The tracheostomy collar is connected to the breathing tube in a tracheostomy incision in the throat through which oxygen is given. While initial placement of a tracheostomy tube is typically performed in a medical care facility, the patient is often discharged with the tube and collar still in place. This means that care of the tracheostomy—including cleaning the incision area and replacing the collar as needed—often falls to the patient's caregiver(s), particularly when the patient is an infant. Existing collars, particularly for infants, are not easy to apply, are difficult to change and do not fit well. The child's small size, frequent movements, and inability to assist can add to the difficulty of providing care. Also, based on current fitting design and attachment tabs, the current collars can cause pain and lead to significant skin irritations, even open wounds on the skin.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1A is a perspective view of the outward-facing side of a collar apparatus in accordance with an embodiment.

FIG. 1B is a perspective view of the inward-facing side of the collar apparatus of FIG. 1A.

FIG. 2A is a view illustrating a use of the collar apparatus of FIG. 1A with a human subject, demonstrating certain aspects of features of the collar apparatus.

FIG. 2B is another view illustrating a use of the collar apparatus of FIG. 1A with a human subject.

FIG. 3A is a perspective view of the outward-facing side of a collar apparatus in accordance with an embodiment.

FIG. 3B is a perspective view of the inward-facing side of the collar apparatus of FIG. 3A.

FIG. 4 is a perspective view of a selected portion of a collar apparatus in accordance with an embodiment.

FIG. 5A through FIG. 5E each show a side view (left) and top view (right) of a fastener element for a collar apparatus according to an embodiment.

In the following detailed description, reference is made to the accompanying drawings which form a part hereof and, in which are shown by way of illustration, specific embodiments of the disclosure that may be practiced. These embodiments are described in sufficient detail to enable those of ordinary skill in the art to practice the invention, and it is to be understood that other embodiments may be utilized, and that structural, logical, and electrical changes may be made within the scope of the disclosure. From the following descriptions, it should be understood that components of the embodiments as generally described and illustrated in the figures herein could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

### DETAILED DESCRIPTION

Current tracheostomy collars (often called “trach collars”) for patients (including infants) have a number of issues:
1. It can be very difficult to get the collar to the appropriate tightness on the first attempt.
2. The collar commonly pinches the patient's skin when the collar is placed.
3. Collars may be too long for patients that have a small diameter neck (e.g. infant), in which case the trach collar has to be customized by cutting the length of the collar and fasteners. When customized by cutting, the fasteners can result in limited contact on the collar which results in a tenuous connection.
4. The anterior fasteners are difficult to thread through the opening on the end of the tracheostomy tube flange.
5. Placement of the fasteners have a potential to cause a pressure injury on the patient's anterior neck. Hook and loop or hook and hook fastener material on the back of the collar irritates the patient.
6. The posterior connector tab on the trach collar is difficult to lift and sometimes repeated adjustment of this connector tab is required to obtain the correct length of the trach collar for essential securement of the trach collar. The connector tabs are often too “strong” and cause difficulty in releasing when repeatedly attaching/reattaching to adjust the collar.
7. Repeated adjustment of the collar by the caregiver to obtain the correct length slows the process of the tracheostomy collar change. Undue time spent in a tracheostomy collar change can result in a variety of patient complications (i.e. decreased oxygenation, increased stress, increased medication requirements, and potential trauma at the trach site).

The inventor has created an improved trach collar that has greater sizing flexibility and has re-engineered the tabs on the collar to be easier to grasp and lift. These and other features can reduce the pain and associated skin irritation that often occur with the existing trach collars.

It should be understood that any reference to an element herein using a designation such as “first,” “second,” and so forth does not limit the quantity or order of those elements, unless such limitation is explicitly stated. Rather, these designations may be used herein as a convenient method of distinguishing between two or more elements or instances of an element. Thus, a reference to first and second elements does not mean that only two elements may be employed there or that the first element must precede the second element in some manner. Also, unless stated otherwise a set of elements may comprise one or more elements.

As used herein, the term “user” refers to a person that actively engages with an apparatus to realize a function or effect of the apparatus, and the term “subject” refers to a person upon whom the apparatus is used. In some cases “user” and “subject” may refer to the same person. Further, while the disclosure may reference use of the trach collar with infants, it will be appreciated that the trach collar can be used with patients of all ages and sizes (e.g., adults, children, and infants).

As used herein with respect to an apparatus to be worn by a human subject (or part of such an apparatus): the term “anterior” indicates element(s) of the apparatus/part that is/are situated on or oriented toward the anterior aspect of the subject's neck when worn; the term “posterior” indicates element(s) of the apparatus that is/are situated on or oriented toward the posterior aspect of the subject's neck when worn; the term “outward” indicates element(s) of the apparatus/part that face away from the surface of the subject's skin; and “inward” indicates element(s) of the apparatus/part that face toward the surface of the subject's skin.

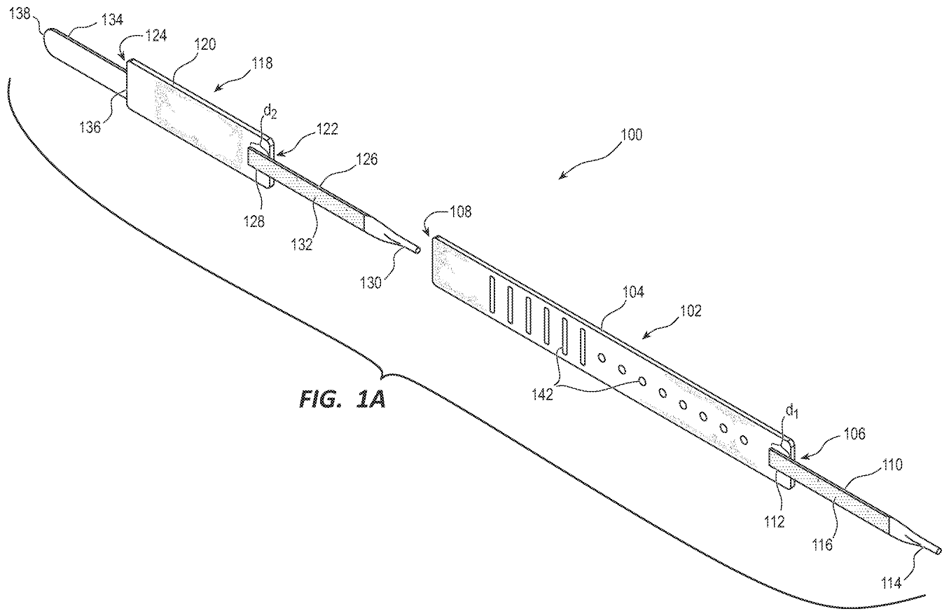

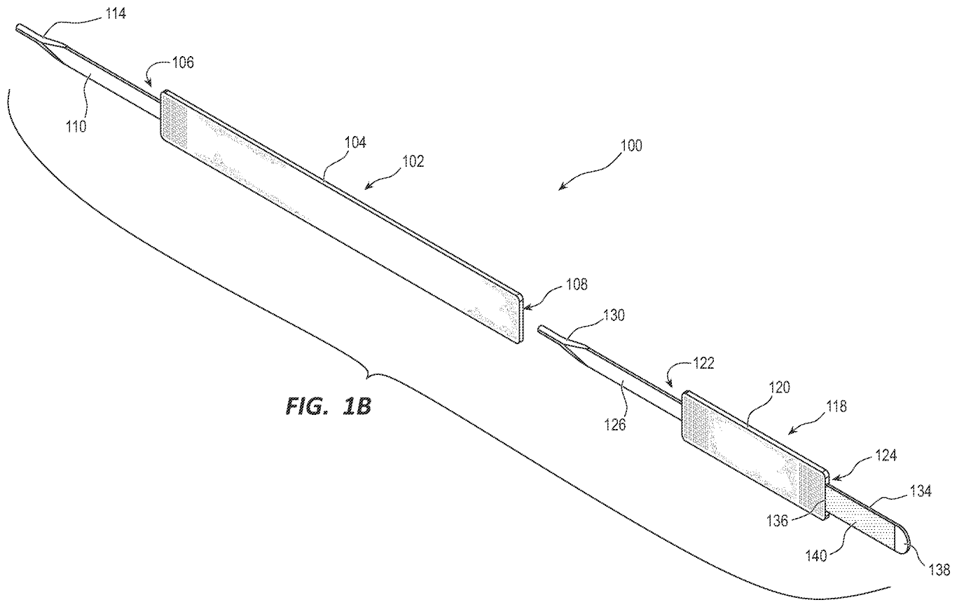

FIG. 1A and FIG. 1B respectively show outward and inward aspects of a collar apparatus 100 for securing a tracheostomy tube to the neck of a subject. As shown the apparatus 100 can comprise a first strap 102 comprising a first strap body 104 defining a length extending between an anterior end 106 and a posterior end 108. The anterior end 106 is configured to engage with an element of a tracheostomy tube, such as a tube flange. As shown in FIG. 1A, a first fastener strip 110 can be anchored adjacent the anterior end 106 for this purpose. The first fastener strip 110 includes a fixed end 112 by which it is anchored to the anterior end 106, and a free end 114 that is configured for engaging and securing the tracheostomy tube through manual manipulation by a user.

In some embodiments, the first fastener strip 110 has a shape (e.g., elongate) that allows it to pass through an opening on a tube flange to facilitate connecting the anterior end 106 to the tube. The free end 114 of the first fastener strip 110 can also be narrow to ease threading through the opening of the tube flange during assembly and use. The free end 114 can also provide a tactile cue to the user's fingers, allowing the user to locate the free end 114 by touch and manipulate the first fastener strip 110. The first fastener strip 110 may be further configured to be secured to the first strap body 104 while engaged with the tube flange to accomplish said connection. For example, the first fastener strip 110 can include a securing surface 116 configured to removably adhere to the first strap body 104. The securing surface 116 can include, without limitation, a releasable adhesive such as a releasable glue, or a mechanical fastener such as a hook and loop or hook and hook fastener. In an embodiment, the securing surface 116 includes a hook and loop or hook and hook fastener component that adhesively engages with an outward surface of the first strap body 104.

The apparatus 100 can further comprise a second strap 118 comprising many or all of the same features discussed above with respect to the first strap 102. That is, the second strap 118 can comprise a second strap body 120 defining a length extending between an anterior end 122 and a posterior end 124. As with the first strap 102, the anterior end 122 can be configured to engage with an element of a tracheostomy tube, such as the tube flange, via a second fastener strip 126 that is anchored adjacent the anterior end 122 by a fixed end 128. The second fastener strip 126 can further include a free end 130 configured to engage the tracheostomy tube for securing the tube to the anterior end 122. This can include a shape such as described above with respect to the first fastener strip 110. The free end 130 can also provide a tactile cue to the user's fingers, allowing the user to locate the free end 130 by touch and manipulate the second fastener strip 126. The second fastener strip 126 can similarly include a means for removably adhering to the second strap body 120, for example a securing surface 132 such as described above.

Through at least the features described above the collar apparatus is operable such that the anterior ends of the strap bodies cooperate in connecting the collar apparatus to a tracheostomy tube placed in the trachea of a subject. The strap bodies have a combined length such that each can extend around the neck of the subject and be mutually connected at or near their posterior ends, forming a closed loop surrounding the neck. In some embodiments the strap bodies are of unequal length. In some embodiments, as illustrated in FIGS. 1A and 1B, the length of the second strap body 120 is less than the length of the first strap body 104. An aspect of this arrangement is that when the collar is in place around a subject's neck, the posterior connection of the strap bodies can be positioned either on a side of the subject's neck or the back of the neck. In particular embodiments, the length of the second strap body is less than about 75% of the length of the first strap body. In a particular embodiment, the length of the second strap body is less than about 50% of the length of the first strap body.

The foregoing can be further accommodated by the first strap body having a length that is greater than one-half the circumference of the neck. In particular embodiments, the length of the first strap body is about 60% to about 95% of a circumference of the neck of the subject. In a particular embodiment, the length of the first strap body is about 70% to about 85% of the circumference of the neck of the subject.

As shown in FIG. 1A and FIG. 1B, in some embodiments the second strap body 120 can include a connector tab 134 configured to facilitate connecting the posterior end 124 of the second strap body 120 to the first strap body 104. The connector tab 134 can comprise a fixed end 136 anchored to the posterior end 124 and also a free end 138 by which the connector tab 134 can be manipulated by a user to connect or disconnect the two strap bodies. In some embodiments, the connector tab 134 includes a securing surface 140 configured to releasably adhere to the first strap body 104. The securing surface 140 can include, without limitation, a releasable adhesive such as a releasable glue, or a mechanical fastener such as a hook and loop or hook and hook fastener. In an embodiment, the securing surface 140 includes a hook and loop or hook and hook fastener component that adhesively engages with an outward surface of the first strap body 104. In some embodiments, the connector tab may include plural securing surfaces. For example, the connector tab may comprise one securing surface on each of two opposite sides of the connector tab. This may allow a component of the apparatus, e.g., a strap body, to adhesively engage with either side of the connector tab. In a particular example, each securing surface may include a hook and loop type fastener, where one securing surface comprises hooks and the opposing surface comprises loops.

In some embodiments, the securing surface 140 does not involve or include the free end 138 of the connector tab 134. Stated differently, when the strap bodies are connected to each other via the connector tab 134, the free end 138 does not adhere to the first strap body 104 and is not involved in the connection. This feature can facilitate easier manipulation of the connector tab 134 by a user, as the user can grasp the free end 138 to connect and disconnect the strap bodies, e.g., while putting on and removing the collar, or to disconnect and reconnect the strap bodies when adjusting the fit of the collar. Also, by remaining unadhered from the first strap body 104, the free end 138 provides a tactile cue to the user's fingers, allowing the user to locate the free end 138 by touch and manipulate the connector tab 134 without a direct line of sight to this region of the collar.

In some embodiments, at least one of the strap bodies include one or more sizing indicia situated to indicate the relative position of the strap bodies when they are connected by the connector tab. An example of this feature is illustrated by the embodiment shown in FIG. 1A, where a number of sizing indicia 142 are displayed on the outward surface of the first strap body 104. In certain embodiments, these indicia 142 can be positioned and spaced to represent particular collar circumferences resulting from various relative positions of the strap bodies. In an aspect, a user can use the sizing indicia 142 as a reference to achieve the same fit on a subject in consecutive uses. In another aspect, a user can use the sizing indicia 142 as a reference when shortening the first strap body 104. For instance, a user can optionally cut off a portion of the first strap body 104 to achieve a smaller fit as needed. In such instances, the sizing indicia 142 can act as a reference when shortening and replacing the first strap body 104, such as to match the length of a prior used strap body 104. The indicia 142 can include any combination of letters, numbers, shapes, and symbols. In various embodiments, the indicia 142 may be colored. In some embodiments, the color(s) of the indicia may be selected to provide added visibility, for example the color(s) may include neon or other bright colors. In some embodiments, the color(s) of the indicia 142 may be selected to indicate sizing, e.g., to provide a color code. In some embodiments, the indicia 142 can further be configured to provide a tactile cue, e.g., the indicia 142 may be raised, recessed and/or textured.

The fastener strips can include further features to enhance ease of use for the user and comfort for the subject. In some embodiments, the free end of at least one of the fastener strips is configured for easier grasping by the user. For example, the free end can include a structure to provide enhanced graspability. One example of this is shown in FIG. 1A and FIG. 1B, in which free end 114 and free end 130 each is shaped to form an aglet. This shape can also facilitate threading the free end 114, 130 through the openings in the tube flange as previously discussed.

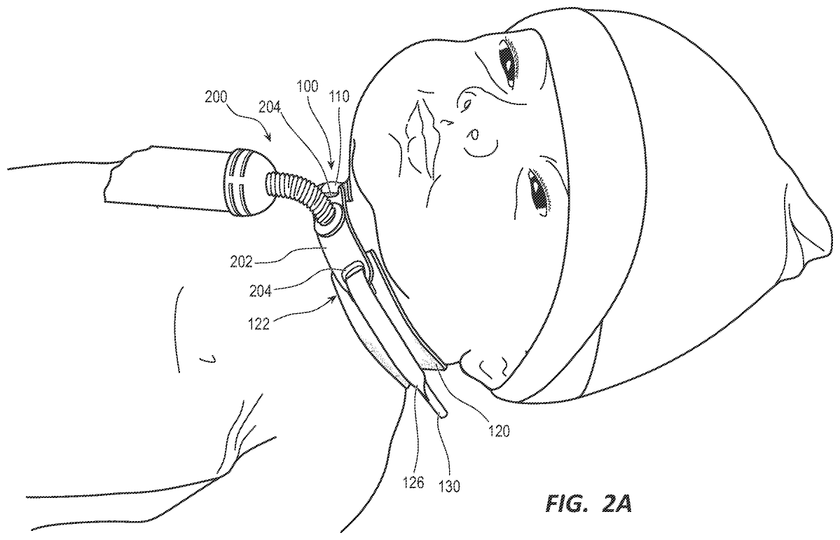

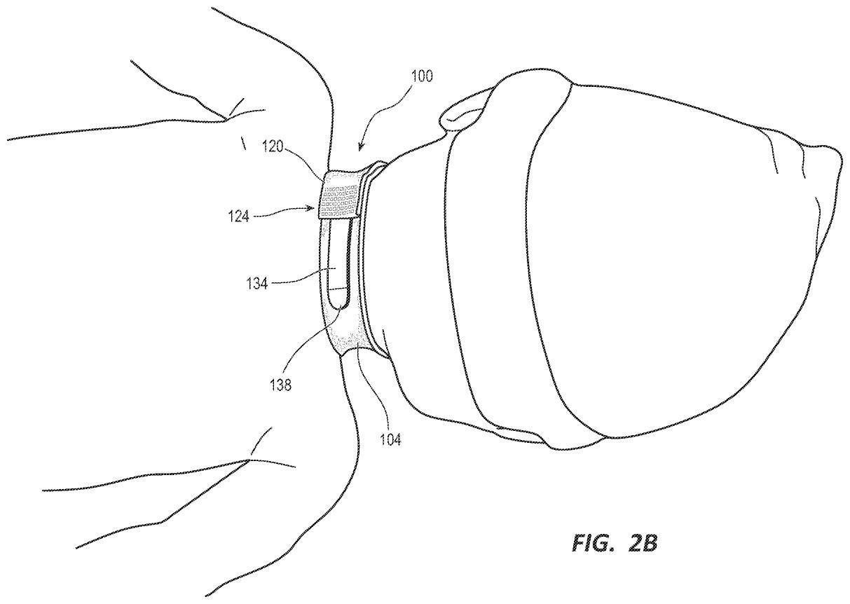

In some embodiments, at least one of the fastener strips is anchored to the corresponding strap body at a distance from the anterior end. Stated another way, this resets the fastener strip's position, so it is anchored in more of a medial direction on the strap body. This is illustrated in the view shown in FIG. 1A, where the first fastener strip 110 is anchored by its fixed end 112 to the first strap body 104 adjacent to the anterior end 106 at a distance di and similarly the second fastener strip 126 is anchored by its fixed end 128 to the second strap body 120 adjacent to the anterior end 122 at a distance dz. In certain embodiments, at least one distance is about 0.1 inches to about 0.5 inches from the anterior end. In an embodiment, the distance is about 0.25 inches. As shown in FIG. 2A and further discussed below, having the fastener strip 110, 126 anchored at a distance di, dz from the anterior end 106, 122 can enable the ends of the tube flange (which may comprise a rigid plastic material) to rest upon the strap body 104, 120, providing comfort and/or minimizing damage to the subject's skin.

The first strap body 104 and second strap body 120 can comprise materials suitable for providing secure positioning of a tracheostomy tube while remaining comfortably in place around the neck for extended periods of time. In certain embodiments, the materials are flexible, such as flexible woven or nonwoven fabrics. In some embodiments, the strap bodies comprise a material that is suited for adhesion with the securing surfaces of the fastener strips and/or the connector tab. For example, when said securing surfaces comprise a hook and loop or hook and hook fastener component, the strap bodies can comprise a complementary hook and loop or hook and hook fastener component. In some embodiments, the strap body can comprise a permeable material that wicks moisture away from the skin. The thickness of the strap body can also be designed to aid in wicking moisture, such as from about 0.1 to about 0.3 inches in thickness. Other thicknesses can also be used.

In some embodiments, various components of the straps can comprise antimicrobial materials or material to which an antimicrobial treatment has been applied. In some embodiments, the antimicrobial material or treatment may comprise silver, for example a silver salt, colloid, or complex. For example, the straps can comprise a polymer matrix within which silver nanoparticles are embedded. In another example, a strap can comprise a textile material including antimicrobial fibers and/or to which a silver-based coating has been applied. Other types of antimicrobial materials can also be used, including other metal (e.g., copper) and non-metal antimicrobial (e.g., organosilanes, quaternary ammoniums, antimicrobial peptides, antimicrobial enzymes, chitosan, etc.) materials.

A method of manufacturing a tracheostomy collar as described herein can comprise positioning a first fastener strip on an anterior end of a first strap body, where the first fastener strip is configured to engage with a first tube flange of a tracheostomy tube; positioning a second fastener strip on an anterior end of a second strap body, where the second fastener strip is configured to engage with a second tube flange of the tracheostomy tube; securing a connector tab to a posterior end of the first strap body, where the connector tab is configured to removably connect the posterior end of the first strap body with a posterior end of the second strap body; and placing sizing indicia (e.g., uniformly or non-uniformly spaced) on an outer surface of the posterior end of the second strap body.

A collar apparatus as described herein can be used in a method of securing a tracheostomy tube equipped with a tube flange to a subject having a tracheostomy into which the tube is inserted. In some embodiments, such use comprises securing the anterior end of the first strap body to an end of the tube flange using the first fastener strip; securing the anterior end of the second strap body to an opposite end of the tube flange using the second fastener strip; extending the first strap body and the second strap body past opposite sides of a neck of the subject so that the neck is encircled thereby; and connecting the posterior end of the second strap body to the first strap body to secure the apparatus around the neck with a selected tightness.

Various aspects of some of the features of the collar discussed above are further illustrated in FIG. 2A and FIG. 2B, which show an example of use of the collar apparatus 100 in securing a tracheostomy tube assembly 200 to the neck of an infant. It will also be appreciated that the collar apparatus 100 can be used with older patients (e.g., children and adults). As shown in the more frontal view of FIG. 2A, the first fastener strip 110 and second fastener strip 126 connect the first strap body 104 (not visible) and second strap body 120 respectively to the tube flange 202 each by being threaded through one of the openings 204 and then adhered to the strap body to which it is anchored. This process is facilitated by the aglet free ends 114 (not visible) and 130.

FIG. 2A also shows an effect of anchoring of each fastener strip at a distance from the anterior end of its corresponding strap body. Noting second fastener strip 126 by way of example, it can be seen that repositioning the fastener strip in a medial direction on the collar allows a length of the anterior end 122 to rest beneath at least a portion of the opening 204 and the terminal end of the tube flange 202. This can decrease the risk of trauma to the skin and/or increase comfort.

FIG. 2B shows an example of the operation of the connector tab 134 in connecting the posterior end 124 of the second strap body 120 to the first strap body 104 to form a closed loop fitted to the neck of the subject. The free end 138 of the connector tab 134 provides an unadhered section that can be readily grasped to disconnect and reconnect the strap bodies as needed to achieve the desired fit.

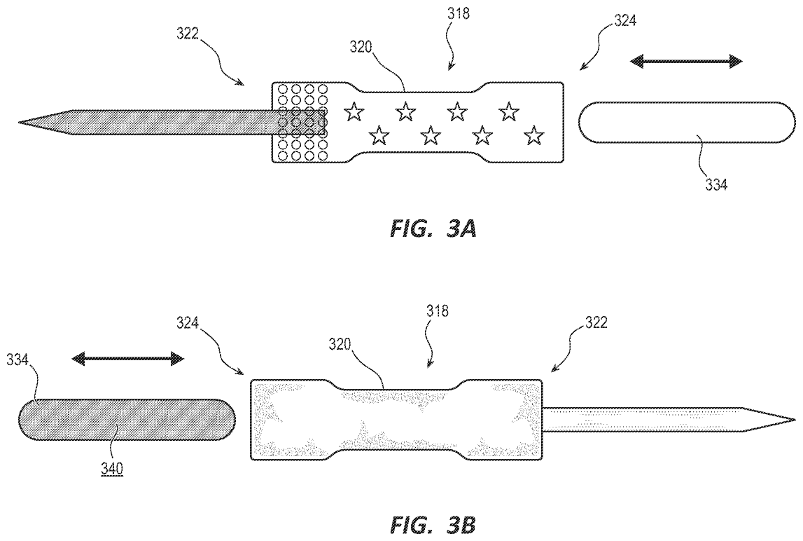

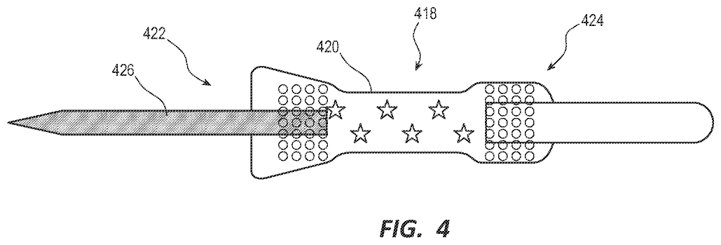

Additional configurations and features are encompassed by the present disclosure. While FIGS. 3A-4 show one of two straps that may form part of a collar apparatus as described herein, the accompanying description applies to corresponding features in both straps. As shown in FIGS. 3A and 3B a collar apparatus for securing a tracheostomy tube to the neck of a subject can comprise a second strap 318 comprising a second strap body 320 extending between an anterior end 322 and a posterior end 324. A connector tab 334 having a securing surface 340 can be configured to facilitate connecting the posterior end 324 of the second strap body 320 to a first strap body (not shown). As shown in FIGS. 3A and 3B, rather than being fixedly attached to either the first strap body or the second strap body 320, the connector tab 334 may be configured for removable attachment with respect to both. Stated in different terms, the position of the connector tab 334 when connecting the two strap bodies can vary relative to each the strap body posterior ends. Such a configuration can aid in sizing the strap 318 and/or collar apparatus.

As shown in FIG. 4, a collar apparatus in accordance with another embodiment can comprise a second strap 418, where said second strap 418 comprises a second strap body 420 with an anterior end 422. As discussed above in reference to FIGS. 1A and 1B, a fastener strip 426 is provided for engaging with an element of a tracheostomy tube, such as a tube flange, where the fastener strip 426 is anchored to the strap body 420 at a distance from the anterior end 422. As illustrated in FIG. 4, the strap body 420 may vary in width along its length. For example, the strap body 420 may be wider at one or both ends than it is in a middle region. As illustrated in FIG. 4, the strap body 420 may increase in width toward its anterior end 422. This can provide a larger area on which the ends of a tube flange may rest during use, providing additional protection to the subject's underlying skin. In accordance with the above embodiment, a first strap body (not shown) may vary in width along its length. For example, the first strap body may be wider at one or both ends than it is in a middle region. The first strap body may increase in width toward its anterior end.

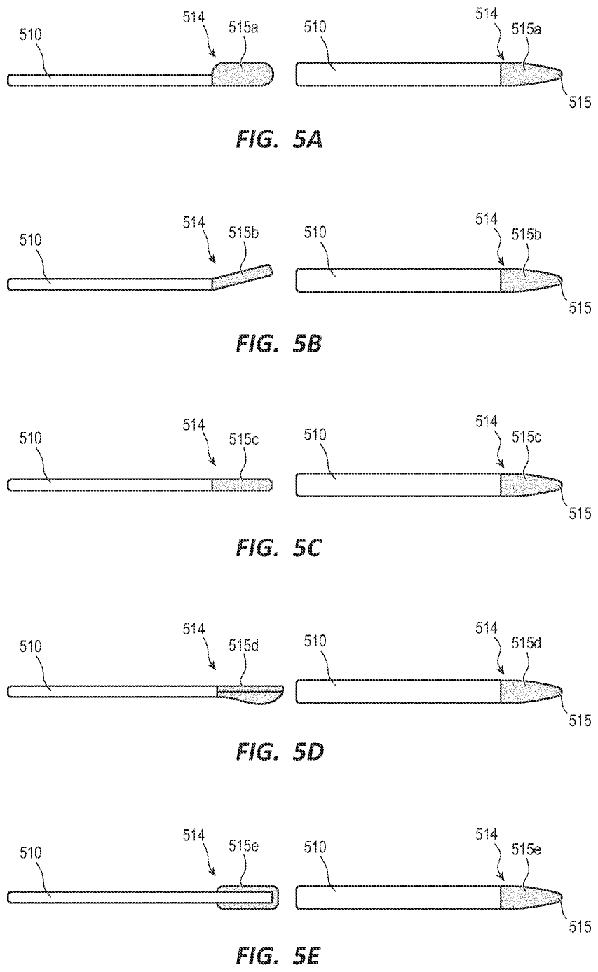

A fastener strip included in a collar apparatus for engaging a tube flange can have a free end configured to be fed through a feature of the tube flange available for that purpose, e.g. an eyelet. The free end can further be configured to be readily grasped by a user, as discussed above. FIGS. 5A-5E each show fastener strips 510 having free ends 514 with various structures that provide this function. FIG. 5A shows a tip structure 515a that may be formed, e.g., by folding a terminal section of the free end 514 over onto itself and dipping it into glue or another adhesive. The resulting tip structure 515a may be further coated with a material to increase graspability, such as natural or synthetic rubber, or another polymeric material. FIG. 5B shows a tip structure 515b that may be formed by crimping or stamping a terminal section of the free end 514 to alter its shape or otherwise form a demarcation between the free end 514 from the rest of the fastener strip 510. The resulting structure 515b may be further coated with a material to increase graspability. FIG. 5C shows a tip structure 515c that may be formed by overlaying a heat shrinkable material over a terminal section of the free end 514 and applying heat to form an aglet tip. FIG. 5D shows a tip structure 515d that may be formed by placing the free end 514 within a molded structure. For example, a molded tip may be provided in two or more pieces that can be assembled around a terminal section of the free end 514. FIG. 5E shows a tip structure 515e that may be formed by gluing or otherwise attaching a piece of material to a terminal section of the free end 514. The material may be selected to increase graspability. In some embodiments, the material is a textured material.

In the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

What is claimed is:

1. An apparatus for securing a tracheostomy tube to the neck of a subject, comprising;
a first strap comprising:
a first strap body defining a length extending from an anterior end to a posterior end; and
a first fastener strip located at the anterior end of the first strap body and comprising:
a fixed end anchored adjacent said anterior end;
a free end configured to pass through an opening of a tracheostomy tube flange; and
a securing surface configured to removably adhere to the first strap body; and
a second strap comprising:
a second strap body defining a length extending from an anterior end to a posterior end;
a second fastener strip located at the anterior end of the second strap body and comprising:
a fixed end anchored adjacent said anterior end;
a free end configured to pass through an opening of a tracheostomy tube flange; and
a securing surface configured to removably adhere to the second strap body; and
a connector tab located at the posterior end of the first or second strap body and comprising a securing surface configured to removably adhere to a strap body,
wherein the length of the second strap body is less than the length of the first strap body.

2. The apparatus of claim 1, wherein the free end of at least one of the first fastener strip and the second fastener strip includes a tip structure to facilitate grasping of the free end.

3. The apparatus of claim 2, wherein the tip structure is formed by folding or crimping said free end.

4. The apparatus of claim 2, wherein the tip structure includes a rubber or polymeric coating.

5. The apparatus of claim 2, wherein the tip structure comprises a molded construct attached to said free end.

6. The apparatus of claim 2, wherein the tip structure comprises a textured material attached to said free end.

7. The apparatus of claim 1, wherein the length of the second strap body is less than about 75% of the length of the first strap body.

8. The apparatus of claim 1 wherein the securing surface of at least one of the first fastener strip and the second fastener strip comprises a hook and loop fastener component.

9. The apparatus of claim 1, wherein the connector tab further comprises:
a fixed end anchored to the posterior end of the first or second strap body; and
a free end,
wherein the securing surface of the connector tab does not include the free end of the connector tab.

10. The apparatus of claim 1, wherein the connector tab is configured to removably adhere to both the first strap body and the second strap body by the securing surface.

11. The apparatus of claim 1, wherein the connector tab further comprises a second securing surface, and wherein the securing surface and the second securing surface are on opposite faces of the connector tab.

12. The apparatus of claim 1, wherein in at least one of the first strap and the second strap the fixed end of the fastener strip is anchored to the strap body at a distance of about 0.1 inches to about 0.5 inches from the anterior end.

13. The apparatus of claim 1, further comprising one or more sizing indicia disposed on the first strap body.

14. The apparatus of claim 13, wherein the sizing indicia are selected from one or more of letters, numbers, shapes or symbols, or wherein the sizing indicia are raised, recessed, colored or textured.

15. The apparatus of claim 1, wherein the length of the first strap body is about 60% to about 95% of a circumference of the neck of the subject.

16. The apparatus of claim 1, wherein at least one of the first strap body and the second strap body is wider at an end than in a middle region.

17. The apparatus of claim 1, wherein at least one of the first strap body and the second strap body includes an antimicrobial material.

18. A method of manufacturing a tracheostomy collar, the method comprising:
positioning a first fastener strip on an anterior end of a first strap body, the first fastener strip configured to engage with a first tube flange of a tracheostomy tube;
positioning a second fastener strip on an anterior end of a second strap body, the second fastener strip configured to engage with a second tube flange of the tracheostomy tube;
securing a connector tab to a posterior end of the first strap body, the connector tab configured to removably connect the posterior end of the first strap body with a posterior end of the second strap body; and
placing uniformly spaced sizing indicia on an outer surface of the posterior end of the second strap body.

19. An apparatus for securing a tracheostomy tube to the neck of a subject, comprising;
a first strap comprising:
a first strap body defining a length extending from an anterior end to a posterior end, the first strap body comprising one or more sizing indicia; and
a first fastener strip located at the anterior end of the first strap body and comprising:
a fixed end anchored adjacent said anterior end;
a free end configured to pass through an opening of a tracheostomy tube flange; and
a securing surface configured to removably adhere to the first strap body; and
a second strap comprising:
a second strap body defining a length extending from an anterior end to a posterior end;
a second fastener strip located at the anterior end of the second strap body and comprising:
a fixed end anchored adjacent said anterior end;
a free end configured to pass through an opening of a tracheostomy tube flange; and
a securing surface configured to removably adhere to the second strap body; and
a connector tab located at the posterior end of the first or second strap body and comprising a securing surface configured to removably adhere to a strap body,
wherein the length of the second strap body is less than the length of the first strap body.

20. The apparatus of claim 19, wherein in at least one of the first strap and the second strap the fixed end of the fastener strip is anchored to the strap body at a distance of about 0.1 inches to about 0.5 inches from the anterior end.
